(19) United States
(12) Patent Application Publication
(10) Pub. No.: US 2006/0019304 A1
(43) Pub. Date: Jan. 26, 2006

(54) Simultaneous analysis of multiple genomes

(76) Inventors: Paul Hardenbol, San Francisco, CA (US); George Karlin-Neumann, Palo Alto, CA (US); Thomas D. Willis, San Francisco, CA (US); Maneesh Jain, San Francisco, CA (US); Nicholas Naclerio, Menlo Park, CA (US)

Correspondence Address: Chief Intellectual Property Counsel;Affymetrix, Inc., 3380 Central Expressway, Santa Clara, CA, 95051, US

(21) Appl. No.: 11/189,486

(22) Filed: Jul. 25, 2005

Related U.S. Application Data
(60) Provisional application No. 60/591,287, filed on Jul. 26, 2004.

(51) Int. Cl. C12Q 1/68; G06F 19/00

(52) U.S. Cl. 435/6; 702/20

(57) ABSTRACT

The invention provides methods for multiplexing readouts from multiple hybridization-based assays that each comprise one or more hybridization or annealing steps and one or more enzymatic processing steps. In one aspect, the invention permits simultaneously analysis of a plurality of genomes by separately hybridizing a set of probes with the different genomes to form sets of probe-genome complexes in separate reaction mixtures that are combined and enzymatically treated to form amplifiable probes. From such amplifiable probes, labeled probes are produced so that for each different locus of each different genome there is a unique labeled probe, which are then specifically hybridized to their respective complements on a microarray. In another aspect, labeled oligonucleotide tags are produced from amplifiable probes. The invention is useful in applications of multiplexed hybridization-based assays for measuring characteristics of genomic samples taken from many different individuals. By conducting hybridization steps separately on samples different individuals then combining them for enzymatic processing, one takes advantage of natural reaction rate differences between hybridization reactions and enzymatic reactions to enable analysis of products of multiple assays on a single readout platform.

This application claims priority to and from U.S. provisional patent application Ser. No. 60/591,287 filed 26 Jul. 2004, which application is incorporated by reference in its entirety.

### FIELD OF THE INVENTION

The invention relates generally to methods genetic analysis using high-density readout platforms, such as microarrays, and more particularly, to methods for multiplexing hybridization-based assays so that results from separate assays can be analyzed on a single readout platform.

### BACKGROUND

Genomic material is complex and thus individual sequences exist at relatively low concentrations in a mixture. As a result of this, at a given probe concentration there is a finite rate at which the probe sequences will hybridize to the genomic templates. Probe concentrations can be adjusted to some extent to vary this rate, but as probe complexity is increased, for example, when highly multiplexed assays are used, such flexibility is greatly reduced. By contrast, enzymes that recognize the hybridized structures often have strong binding affinities and thus can be made to process probe-genome complexes into amplification templates in a much smaller time (seconds to minutes).

Several hybridization-based assays are available for analyzing DNA that include a hybridization step in which a probe is hybridized to a genomic template and an enzyme processing step in which a template-driven reaction produces an amplification template that can be amplified using standard methods, e.g. Syvanen, Nature Genetics Supplement, 37: S5-S10 (2005). Amplicons produced from such amplification templates can then be read out in parallel by hybridizing sequences present on the labeled probes to their respective complements on a solid phase support, such as a microarray.

Since microarrays can be efficiently produced that contain millions of features and since studies of complex genetic processes frequently require the analysis of samples from many different genomes, it would be an advantage in the above methods if different genomes partially processed in separate reactions could be combined and read out on a single microarray. Since many studies do not require full use of an entire microarray for any one sample, this would decrease the cost and increase the throughput of such methods.

### SUMMARY OF THE INVENTION

The present invention provides methods for multiplexing readouts from multiple hybridization-based assays that each comprise one or more hybridization or annealing steps and one or more enzymatic processing steps. In particular, the invention provides a method for simultaneously analyzing a plurality of genomes to obtain sequence information at one or more loci in each genome by carrying out the following steps: (a) providing for each genome a set of probes, each probe within a set being specific for a locus of the genome; (b) separately hybridizing each set of probes with its respective genome to form probe-genome complexes in separate reaction mixtures; (c) combining the separate reaction mixtures and enzymatically treating the probe-genome complexes to form amplifiable probes; (d) amplifying and labeling the amplifiable probes to form labeled probes, so that for each different locus of each different genome there is a unique labeled probe; and (e) specifically hybridizing the labeled probes to their respective complements on a microarray so that the presence or absence of a labeled probe specifically hybridized to the microarray is indicative of sequence information of each of the one or more loci of each genome in the plurality. In one aspect, each of the probes contains an oligonucleotide tag.

The present invention is useful in applications of multiplexed hybridization-based assays for measuring characteristics of genomic samples taken from many different individuals. By conducting hybridization steps separately on different individual samples then combining them for enzymatic processing, one takes advantage of natural reaction rate differences between hybridization reactions and enzymatic reactions to enable analysis of products of multiple assays on a single readout device, such as a microarray, set of microbeads, or the like.

### BRIEF DESCRIPTION OF THE DRAWINGS

FIGS. 1A-1B diagrammatically illustrate the operation of one embodiment of the invention.

FIGS. 1C-1E illustrate different ways in which information from different loci of different genomes can be encoded by oligonucleotide tags and fluorescent labels.

FIGS. 2A-2B diagrammatically illustrate the use of multiple sets of molecular inversion probes for genotyping different genomes wherein a single readout device is used in accordance with the invention.

### DEFINITIONS

Terms and symbols of nucleic acid chemistry, biochemistry, genetics, and molecular biology used herein follow those of standard treatises and texts in the field, e.g. Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” or “addressed” in reference to tag complements means that the nucleotide sequence, or perhaps other physical or chemical characteristics, of a tag complement can be determined from its address, i.e. a one-to-one correspondence between the sequence or other property of the tag complement and a spatial location on, or characteristic of, the solid phase support to which it is attached. Preferably, an address of a tag complement is a spatial location, e.g. the planar coordinates of a particular region containing copies of the tag complement. In other embodiments, probes may be addressed in other ways, e.g. by microparticle size, shape, color, color- or fluorescent ratio, radio frequency of micro-transponder, or the like, e.g. Kettman et al, Cytometry, 33: 234-243 (1998); Xu et al, Nucleic Acids Research, 31: e43 (2003); Bruchez, Jr. et al, U.S. Pat. No. 6,500,622; Mandecki, U.S. Pat. No. 6,376,187; Stuelpnagel et al, U.S. Pat. No. 6,396,995; Chee et al, U.S. Pat. No. 6,544,732; Chandler et al, PCT publication WO 97/14028; and the like.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Complex” means an assemblage or aggregate of molecules in direct or indirect contact with one another. In one aspect, “contact,” or more particularly, “direct contact” in reference to a complex of molecules, or in reference to specificity or specific binding, means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including tempature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable complex as the result of a specific binding event. In one aspect, a hybridization-based assay means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay means a polynucleotide that has a sequence that is capable of forming a stable hybrid (or triplex) with its complement in a target nucleic acid and that is capable of being detected, either directly or indirectly. Hybridization-based assays include, without limitation, assays that use the specific base-pairing of one or more oligonucleotides as target recognition components, such as polymerase chain reactions, NASBA reactions, oligonucleotide ligation reactions, single-base extension reactions, circularizable probe reactions, allele-specific oligonucleotide hybridizations, either in solution phase or bound to solid phase supports, such as microarrays or microbeads, and the like. An important subset of hybridization-based assays include such assays that have at least one enzymatic processing step after a hybridization step. Hybridization-based assays of this subset include, without limitation, polymerase chain reactions, NASBA reactions, oligonucleotide ligation reactions, cleavase reactions, e.g. in Invader® assays, single-base extension reactions, probe circularization reactions, and the like. There is extensive guidance in the literature on hybridization-based assays, e.g. Hames et al, editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay includes circumstances where either probes or target sequences are attached to microbeads such that the attached sequences have substantially the same environment (e.g., permitting reagent access, etc.) as free sequences. In another aspect, hybridization-based assays include immunoassays wherein antibodies employ nucleic acid reporters based on amplification. In such assays, antibody probes specifically bind to target molecules, such as proteins, in separate reactions, after which the products of such reactions (i.e., antibody-protein complexes) are combined and nucleic acid reporters are amplified. Preferably, such nucleic acid reporters include oligonucleotide tags that are converted enzymatically into labeled oligonucleotide tags for analysis on a microarray, as described below. The following exemplary references disclose antibody-nucleic acid conjugates for immunoassays and are incorporated herein by reference: Baez et al, U.S. Pat. No. 6,511,809; Sano et al, U.S. Pat. No. 5,665,539; Eberwine et al, U.S. Pat. No. 5,922,553; Landegren et al, U.S. Pat. No. 6,558,928; Landegren et al, U.S. patent publication 2002/0064779; and the like. In particular, the two latter patent publications by Landegren et al disclose steps of forming amplifiable probes after a specific binding event.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. In one aspect, kits of the invention comprise probes specific for interfering polymorphic loci. In another aspect, kits comprise nucleic acid standards for validating the performance of probes specific for interfering polymorphic loci. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a type of multiplex assay product that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable. Probes immobilized on microarrays include nucleic acids, such as oligonucleotide barcodes, that are generated in or from an assay reaction. Typically, the oligonucleotides or polynucleotides on microarrays are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999); and Fodor et al, U.S. Pat. Nos. 5,424,186; 5,445,934; and 5,744,305. Microarray may comprise arrays of microbeads, or other microparticles, disposed on a planar surface. Such microarrays may be formed in a variety of ways, as disclosed in the following exemplary references: Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. In one format, microarrays are formed by randomly disposing microbeads having attached oligonucleotides on a surface followed by determination of which microbead carries which oligonucleotide by a decoding procedure, e.g. as disclosed by Gunderson et al, U.S. patent publication Ser. No. 2003/0096239.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g. comprising from about 3 to about 100 monomers, and the term “polynucleotide” usually refers to longer polymers, e.g. comprising from about 100 monomers to many thousands of monomers, e.g. 10,000 monomers, or more. Oligonucleotides comprising probes or primers usually have lengths in the range of from 12 to 60 nucleotides, and more usually, from 18 to 40 nucleotdes. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like. Usually nucleosidic monomers are linked by phosphodiester bonds. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Oligonucleotides and polynucleotides may be single stranded or double stranded.

“Oligonucleotide tag” means an oligonucleotide that is attached to a polynucleotide and is used to identify and/or track the polynucleotide in a reaction. Usually, a oligonucleotide tag is attached to the 3′- or 5′-end of a polynucleotide to form a linear conjugate, sometime referred to herein as a “tagged polynucleotide,” or equivalently, an “oligonucleotide tag-polynucleotide conjugate,” or “tag-polynucleotide conjugate.” Oligonucleotide tags may vary widely in size and compositions; the following references provide guidance for selecting sets of oligonucleotide tags appropriate for particular embodiments: Brenner, U.S. Pat. No. 5,635,400; Brenner et al, Proc. Natl. Acad. Sci., 97: 1665-1670 (2600); Shoemaker et al, Nature Genetics, 14: 450-456 (1996); Morris et al, European patent publication 0799897A1; Wallace, U.S. Pat. No. 5,981,179; and the like. In different applications of the invention, oligonucleotide tags can each have a length within a range of from 4 to 36 nucleotides, or from 6 to 30 nucleotides, or from 8 to 20 nucleotides, respectively. In one aspect, oligonucleotide tags are used in sets, or repertoires, wherein each oligonucleotide tag of the set has a unique nucleotide sequence. In some embodiment, particularly where oligonucleotide tags are used to sort polynucleotides, or where they are identified by specific hybridization, each oligonucleotide tag of such a set has a melting temperature that is substantially the same as that of every other member of the same set. In such aspects, the melting temperatures of oligonucleotide tags within a set are within 10° C. of one another; in another embodiment, they are within 5° C. of one another; and in another embodiment, they are within 2° C. of one another. In another aspect, oligonucleotide tags are members of a minimally cross-hybridizing set. That is, the nucleotide sequence of each member of such a set is sufficiently different from that of every other member of the set that no member can form a stable duplex with the complement of any other member under stringent hybridization conditions. In one aspect, the nucleotide sequence of each member of a minimally cross-hybridizing set differs from those of every other member by at least two nucleotides. Such a set of oligonucleotide tags may have a size in the range of from several tens to many thousands, or even millions, e.g. 50 to 1.6×10⁶. In another embodiment, such a size is in the range of from 200 to 40,000; or from 200 to 40,000; or from 200 to 10,000.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g. an allele present in a population at a frequency of fifty percent or greater.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a characteristic of one or more signal generation moieties, or labels, that are measured, detected, and/or counted and that can be converted to a number or value. In one aspect, a readout of an assay is obtained by the use or application of a instrument and/or process that converts assay results on the molecular level into signals that may be detected and recorded. Such instrument or process may be referred to as a “readout device” (or instrument) or “readout process” (or method). A readout can also include, or refer to, an actual numerical representation of such collected or recorded data. For example, a readout of a hybridization assay using a microarray as a readout device collectively refers to signals generated at each feature, or hybridization site, of the microarray and their numerical, graphical, and/or pictorial representations.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as Van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tₘ” is used in reference to “melting temperature.” Melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE INVENTION

The invention provides a method of using a single readout device, such as a high-capacity microarray, to analyze products from multiple hybridization-based assays each directed to analyzing loci in different genomes. In particular, multiplex hybridization-based assays of the invention comprise a hybridization or annealing step in which one or more oligonucleotide components of the assay specifically hybridize to their complementary sequences in one or more target polynucleotides to form recognition structures, referred to herein as “probe-genome complexes,” and an enzymatic processing step in which one or more enzymes operate on one of more of their respective recognition structures to produce amplifiable probes. In one aspect, the hybridization step (or steps) of a plurality of multiplex hybridization-based assays are carried out separately from one another, after which the products of such step are combined in order to carry out one or more enzymatic processing steps. An amplifiable probe is a nucleic acid structure, such as a duplex, circular single stranded DNA, or the like, that can be replicated in whole or in part to produce a probe that is specifically hybridized to its complement on a microarray. Sequence information obtained from hybridization-based assays used with the invention includes the presence, absence, or quantity of a particular nucleic acid sequence in a target polynucleotide, such as a genomic fragment, RNA, cDNA, or the like. In one aspect, sequence information obtained from hybridization-based assays includes the presence or absence of single nucleotide polymorphisms (SNPs), insertions, or deletions at particular genomic loci.

As mentioned above, the hybridization step(s) of hybridization-based assays of the invention are significantly longer than the enzymatic processing step(s). Thus, after probe-genome complexes from multiple hybridization based assays are combined, enzymatic processing takes place before there can be any significant dissociation and spurious re-annealing of probes, that is, re-annealing to target polynucleotides originating from other hybridization-based assays. The relative duration of incubation times for the hybridization step(s) and the enzymatic processing step(s) varies widely and depends of factors well-known to those of ordinary skill in the art, for example, the complexity and concentration of probe and target polynucleotides, salt concentrations, presence or absence of co-factors, presence or absence of hybridization accelerants, temperature, the types of enzymes used in the processing step(s), enzyme reaction conditions, and the like. In one aspect, the incubation time of the hybridization step(s) is greater than 100 times that of the enzymatic processing steps (s). In another aspect, the incubation time of the hybridization step(s) is greater than 10 times that of the enzymatic processing step(s). In another aspect, a hybridization step of a hybridization-based assay requires an incubation time in the range of from a few hours, e.g. 2-4 hours, to several tens of hours, e.g. 24 to 72 hours; in other aspects, such incubation time may be in the range of from 2 to 48 hours, or from 2 to 24 hours. In another aspect, one or more enzymatic processing steps in a hybridization-based assay requires an incubation time in the range of from 1 to 60 minutes; or in another aspect, such steps may require an incubation time in the range of from 1 to 30 minutes.

Enzymatic treatment or processing of probe-genome complexes and/or other reactants or products of hybridization steps can include the use of one or more enzymes of several different activities. Processing enzymes include virtually any enzyme that permits or enhances the ability to distinguish probes that successfully detect their intended target sequences from those that do not. Usually, this is accomplished by selecting enzymes for which probe-genome complexes are substrates and which produce a new or modified structure by enzymatic activity. However, enzymes, particularly nucleases, that recognize and digest probes that do not hybridize, or mis-hybridize, to their target sequences are also within the purview of enzymatic treatments of the invention. In one aspect, enzymatic treatment or processing includes treatment or processing with polymerases, ligases, exonucleases, endonucleases, cleavases, phosphatases, kinases, or the like. In another aspect, enzymatic treatment includes treatment with one or more enzymes whose substrates include a nucleic acid template. In another aspect, such treatment includes treatment with at least one DNA polymerase or at least one DNA ligase in a template-driven reaction.

Multiplexing occurs at two levels in the invention. First, hybridization-based assays are designed to measure characteristics of one or more target polynucleotides, such as genomic fragments, at multiple loci. The number of loci measured in each hybridization-based assay can vary widely, and its upper limit depends on well-known factors, including the capacity of the readout device being employed, trade-offs selected between probe concentrations and reaction times, the genetic characteristic being measured (for example, trait association studies require very large numbers of measurements; genetic identification requires relative few measurements; testing for adverse drug reactions may require a medium number of measurements, e.g. several tens to hundreds), and the like. In one aspect, the number of loci analyzed in each hybridization-based assay is in the range of from a few tens, e.g. 10 to 20, to many thousands, e.g. fifty to a hundred thousand. In another aspect, the number of loci per hybridization-based assay is in the range of from 10 to 40,000; or it is in the range of from 100 to 30,000; or it is in the range of from 100 to 20,000. Second, multiplexing occurs from the combination of assay products from multiple hybridization-based assays for determining multiple signals on a single readout device. Usually, there is a one-to-one correspondence between the number of hybridization-based assay products combined in accordance with the invention and the number of different genomic samples being analyzed; however, the invention comprehends situations where different numbers of samples from different individuals are analyzed by varying numbers of hybridization-based assays, the products of which are combined. The level of such multiplexing depends of the capacity of the readout device and the number of loci measured by each hybridization-based assay. In one aspect, the number of hybridization-based assays that may be multiplexed is simply the capacity of a readout device, e.g. the number of features on a microarray, divided by the number of loci being measured in the hybridization-based assays. For example, in one aspect, a 100 thousand-feature microarray can serve as a readout device for fifty hybridization based assays that each measure the zygosity of one thousand loci, when two features are required for each determination (e.g. homozygous in allele 1, homozygous in allele 2, or heterozygous in allele 1 and 2). As discussed more fully below, particular trade-offs between the use of microarray features, different colored fluorescent dyes, number of loci measured per hybridization-based assay, capacity of a readout device, and the like, are routine design choices for those of ordinary skill in the art.

In one embodiment, an amplifiable probe of the invention comprises at least one oligonucleotide tag that is replicated and labeled to produce a labeled oligonucleotide probe. In such embodiment, labeled oligonucleotide probes are hybridized to a microarray of tag complements for detection. In this embodiment, for each different locus of each different genome there is a unique labeled oligonucleotide tag. That is, the pair consisting of (i) the nucleotide sequence of the oligonucleotide tag and (ii) a label that generates detectable signal are uniquely associated with a particular locus of a particular genome. The nature of the label on an oligonucleotide tag can be based on a wide variety of physical or chemical properties including, but not limited to, light absorption, fluorescence, chemiluminescence, electrochemiluminescence, mass, charge, and the like. The signals based on such properties can be generated directly or indirectly. For example, a label can be a fluorescent molecule covalently attached to an amplified oligonucleotide tag that directly generates an optical signal. Alternatively, a label can comprise multiple components, such as a hapten-antibody complex, that, in turn, may include fluorescent dyes that generated optical signals, enzymes that generate products that produce optical signals, or the like. Preferably, the label on an oligonucleotide tag is a fluorescent label that is directly or indirectly attached to an amplified oligonucleotide tag. In one aspect, such fluorescent label is a fluorescent dye or quantum dot selected from a group consisting of from 2 to 6 spectrally resolvable fluorescent dyes or quantum dots.

Flourescent labels and their attachment to oligonucleotides, such as oligonucleotide tags, are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519. In one aspect, one or more fluorescent dyes are used as labels for labeled target sequences, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5,847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. Labeling can also be carried out with quantum dots, as disclosed in the following patents and patent publications, incorporated herein by reference: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer, and the like.

Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY®R-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000), the disclosure of which is incorporated herein by reference in its entirety.

Other fluorophores available for post-synthetic attachment include, inter alia, Alexa Fluor® 350, Alexa Fluor® 532, Alexa Fluor® 546, Alexa Fluor® 568, Alexa-Fluor® 594, Alexa Fluor® 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, Cy5.5, and Cy7 (Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligos bound to the array. Lakowicz et al., BioTechniques 34: 62-68 (2003).

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivitized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any subfragment thereof, such as an Fab.

Other suitable labels for detection oligonucleotides may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr), or any other suitable label. In one embodiment the following hapten/antibody pairs are used for detection, in which each of the antibodies is derivatized with a detectable label: biotin/α-biotin, digoxigenin/a-digoxigenin, dinitrophenol (DNP)/α-DNP, 5-Carboxyfluorescein (FAM)/α-FAM.

As mentioned above, oligonucleotide tags can be indirectly labeled, especially with a hapten that is then bound by a capture agent, e.g. as disclosed in Holtke et al, U.S. Pat. Nos. 5,344,757; 5,702,888; and 5,354,657; Huber et al, U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g. Molecular Probes).

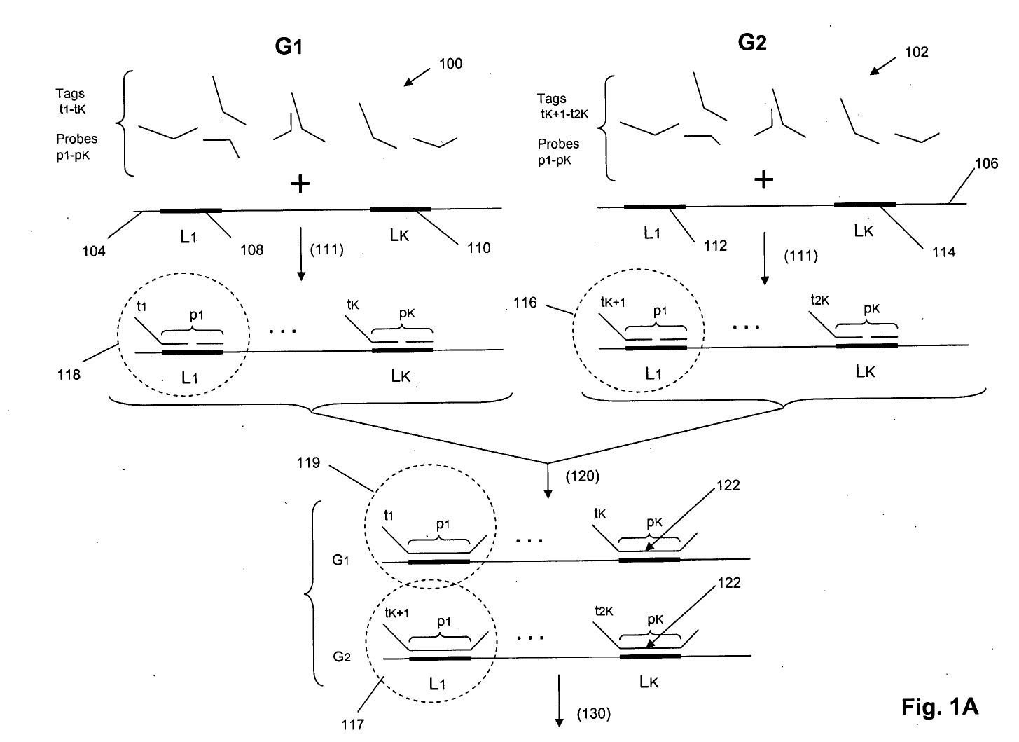

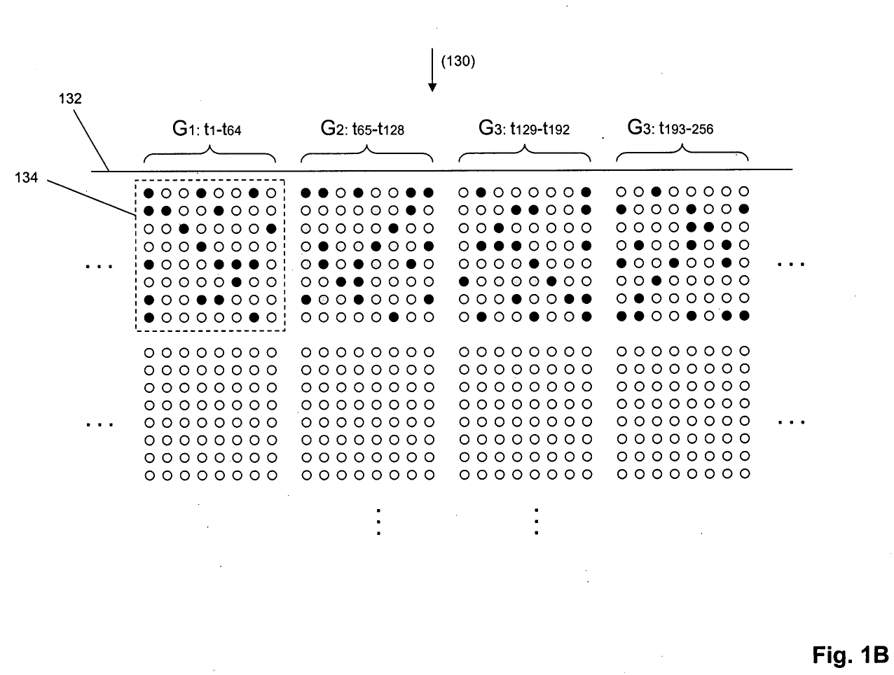

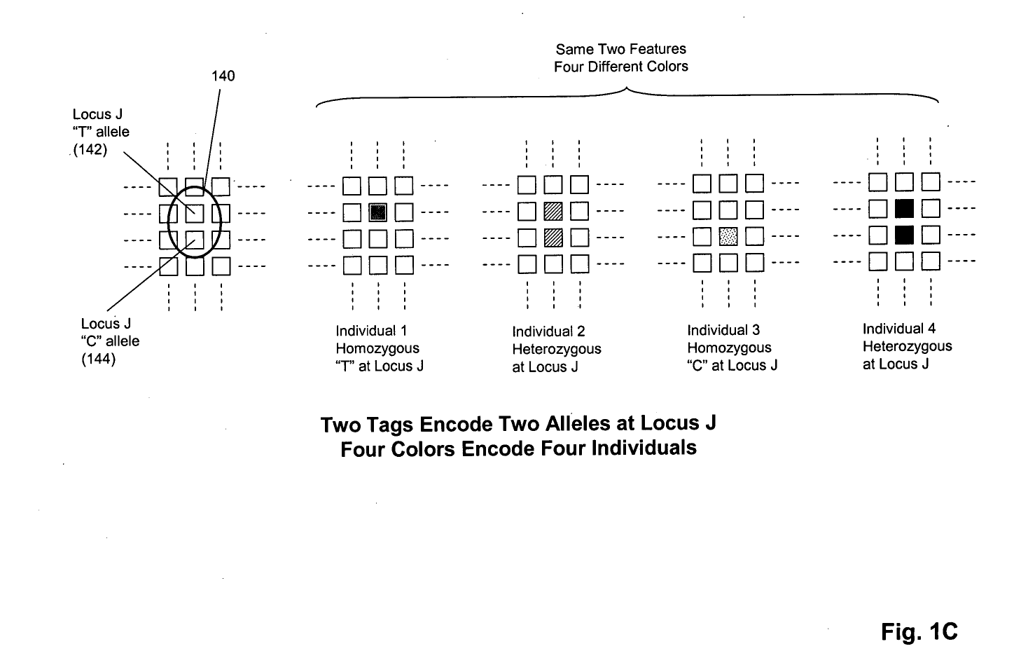

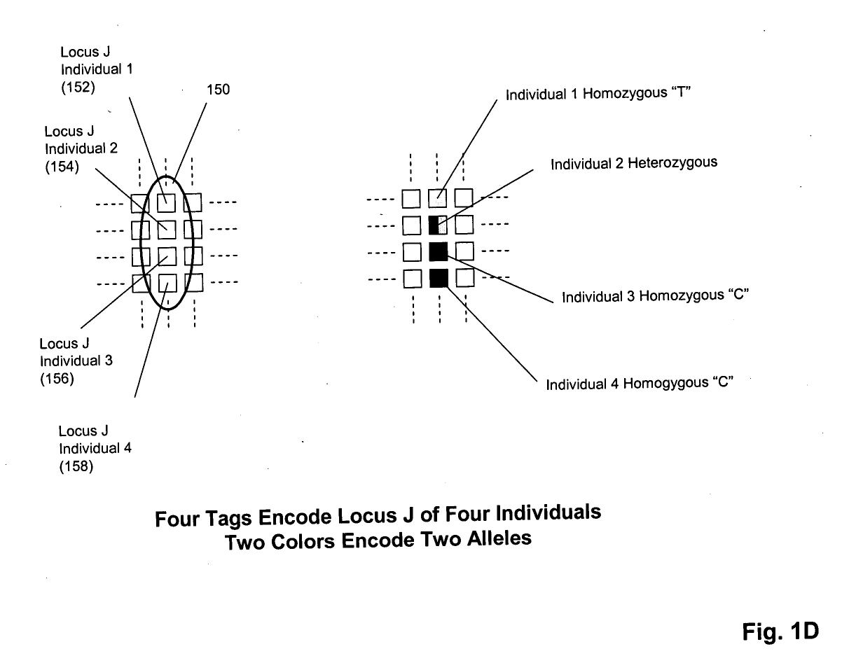

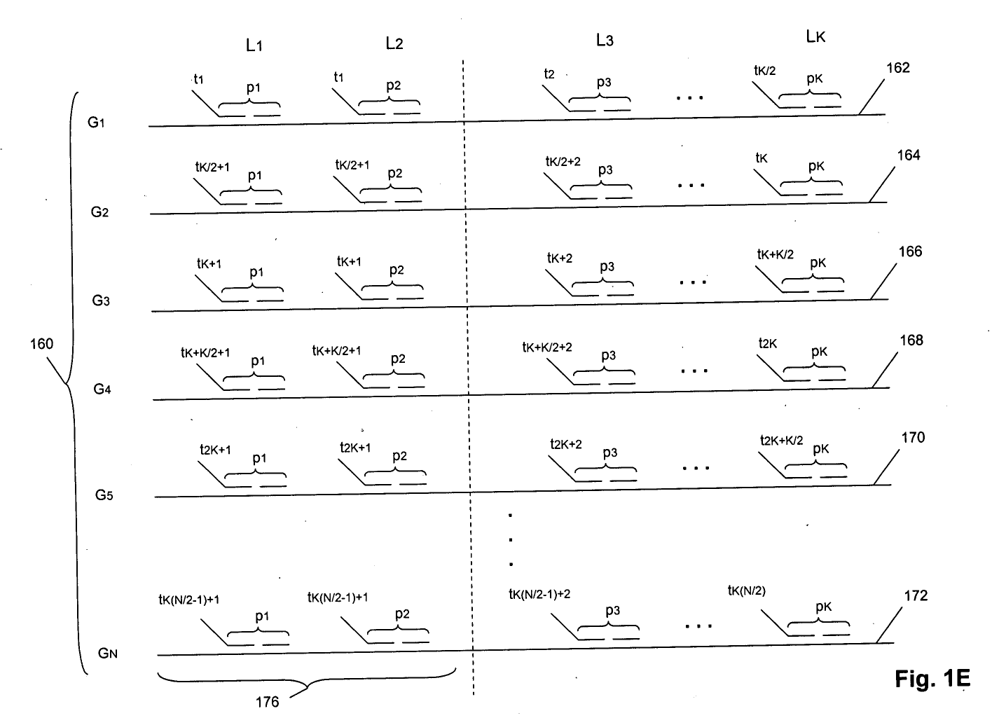

Operation of one embodiment of the invention is illustrated in FIGS. 1A and 1B for a particular hybridization-based assay (one based on the template-driven ligation of two oligonucleotide components). Hybridization-based assays are carried out separately on a sample of DNA from genome G₁ and a sample of DNA from genome G₂. Probes p₁ to p_{K} (100) containing oligonucleotide tags t₁ to t_{K}, respectively, and probes p₁ to p_{K} (102) containing oligonucleotide tags t_{K+1} to t_{2K}, respectively, are combined in separate reaction mixtures with genome G₁ DNA and genome G₂ DNA, respectively, under conditions that permit specific hybridization of such probes to their respective DNA stands (104) and (106) containing target loci, L₁ through L_{K} ((108) to (110) for genome G₁ and (112) to (114) for genome G₂). After an incubation time depending on parameters well-known to those of ordinary skill, e.g. target complexity, probe concentration, temperature, and the like, discussed more fully below, probe-genome complexes form ((118) and (116), respectively) that include recognition structures for enzyme(s) of subsequent step(s). Products of the hybridization step are combined (120) and treated with appropriate enzymes for the particular assay. In the example of FIG. 1A, probes p1 through pK forming perfectly matched duplexes with their respective templates are ligated using a conventional ligase enzyme. Optionally, each up-stream component of probes p₁ through p_{K} can have a nuclease-resistant 3′ end, in which case ligation produces a molecule that is resistant to digestion by 3′-exonucleases. (Or, alternatively, if ligation produces a circular probe, the same result is achieved). In such aspects, an enzymatic treatment step comprises treatment with a ligase followed by treatment with a 3′-exonuclease to produce amplifiable probes. The 3′-exonuclease digests and renders unamplifiable the probes that fail to ligated, for example, because there is no target polynucleotide that permits the formation of a recognition structure for the ligase being employed. After formation of probe-genome complexes (121), ends (122) of the probe components are ligated together whenever both components form perfectly matched duplexes with their respective templates. After such ligation (as exemplified here), oligonucleotide tags of successfully ligated probes are amplified and labeled (124) to produce labeled oligonucleotide tags that are then specifically hybridized to a microarray (132), as illustrated in FIG. 1B. In this figure, for convenience, all the tag complements corresponding to each different genome are shown grouped together in 8×8 sectors, such as that indicated by dashed lines (134). Such grouping is not necessary for practice of the invention, and the respective tag complements may be inter-mixed on a microarray, so long as they have known addresses. In this illustration, four 8×8 sectors are shown with representations of three types of signals that may be collected from each hybridization site. The illustration presumes that each locus has two possible alleles present and that an individual can be homozygous in either one of the alleles, as well as heterozygous in such alleles. Open circles represent a signal from an individual homozygous in a first allele at a particular locus, black-filled circles represent a signal from an individual homozygous in a second allele at a locus, and gray-filled circles represent a signal from an individual that is heterozygous in the possible alleles of a locus.

FIGS. 1C-1E illustrate exemplary ways in which information from assays can be encoded by the addresses of hybridization sites and signal characteristics. Selection of particular embodiments based on these examples may require design trade-offs that are familiar to those of ordinary skill in the art, such as reduction of probe costs versus more sophisticated detection system that can measure emissions from multiple fluorescent dyes. FIG. 1C illustrates a scheme wherein two hybridization sites (142) and (144) contained in oval (140) each contain a distinct tag complement associated with a different allele of a single locus. In this scheme, fluorescent dyes having four different emission bands (represented by gray, cross-hatched, stippled, and black squares, respectively) correspond to four different individuals; hybridization sites (142) and (144) correspond to two different alleles at a single locus, J. In such an embodiment, far fewer probes having different oligonucleotide tags would have to be produced; however, a detection system that could distinguish simultaneously four different emission bands would be required. In this example, the zygosity at a locus in each of four different individuals is determined from the fluorescent emissions of two hybridization sites. FIG. 1D illustrates the scheme discussed above in connection with FIGS. 1A-1B. That is, each different locus of each different individual is assigned a different oligonucleotide tag that may be labeled in one of three ways depending on whether an individual is homozygous in allele 1 or allele 2 or heterozygous is allele 1 and 2. Thus, oval (150) identifies four hybridization sites (152), (154), (156), and (158) corresponding to the same locus J in individuals 1 through 4, respectively, and zygosity of locus J in each of such individuals is determined by the fluorescent emissions of two fluorescent dyes.

FIG. 1E shows still another scheme for encoding signals from hybridization sites. Genomes G₁ through G_{N} (160) are shown as separate lines (162) through (172) each containing loci L₁ through L_{K} in order from left to right. At each locus of each genome, probes are shown hybridized. In this scheme, loci are grouped as pairs (176) wherein each pair of within a genome has the same oligonucleotide tag, and the same pair in a different genome has a different oligonucleotide tag. In such scheme, the zygosity of two two-allele loci is determined by the emissions of four spectrally resolvable dyes. At a single hybridization site, emissions are collected in four channels so that two channels give the zygosity of one locus and two additional channels give the zygosity of a second locus.

As discussed above, amplifiable probes are formed from probes that have been modified in a reaction subsequent to specifically binding to a target genome. The modification permits the probes to be selected, for example, by removal or separation from unmodified probes, by destruction of unmodified probes and/or non-target polynucleotides, or by other such means. Modifications may comprise chemical or enzymatic modification, such as ligation, or extension with a polymerase. In one aspect, probes are modified by ligation so that they form closed circular DNAs. In another aspect, probes are extended by a nucleic acid polymerase to incorporate a modified nucleotide that contains a capture moiety, such as biotin. In another aspect, both of the above modifications are effected by one or more template-driven enzymatic reactions. Exemplary probes include molecular inversion probes, padlock probes, rolling circle probes, ligation-based probes with “zip-code” tags, single-base extension probes, and the like, e.g. Hardenbol et al, Nature Biotechnology, 21: 673-678 (2003); Nilsson et al, Science, 265: 2085-2088 (1994); Baner et al, Nucleic Acids Research, 26: 5073-5078 (1998); Lizardi et al, Nat. Genet., 19: 225-232 (1998); Gerry et al, J. Mol. Biol., 292: 251-262 (1999); Fan et al, Genome Research, 10: 853-860 (2000); International patent publications WO 2002/57491 and WO 2000/58516; U.S. Pat. Nos. 6,506,594 and 4,883,750; and the like, which references are incorporated herein by reference. In one aspect, probes of the invention are molecular inversion probes, e.g. as disclosed in Hardenbol et al (cited above) and in Willis et al, U.S. Pat. No. 6,858,412, which are incorporated herein by reference. In the case of molecular inverstion probes, amplifiable probes are formed by circularizing probes in a template-driven reaction on a target genome followed by digestion with one or more exonucleases of non-circularized polynucleotides, such as target genomes, unligated probe, probe concatatemers, and the like. In another aspect, probes comprise an oligonucleotide tag and a target-specific region that is extended by a polymerase reaction to add a nucleotide with a capture moiety, such as biotin, as disclosed in Fan et al (cited above) and Mao et al, PCT publication WO 02/097113. Amplifiable probes are formed by capturing extended probes on a solid phase support derivatized with a capture agent, e.g. avidinated magnetic microbeads, and separating them from the reaction mixture.

Many different terminator-capture moiety combinations are available. Preferably, dideoxynucleoside triphosphates are used as terminators. In one aspect, capture moieties may be attached to such terminators derivatized with an alkynylamino group, as taught by Hobbs et al, U.S. Pat. No. 5,047,519 and Taing et al, International patent publication WO 02/30944, which are incorporated herein by reference. Preferable capture moieties include biotin or biotin derivatives, such as desbiotin, which are captured with streptavidin or avidin or commercially available antibodies, and dinitrophenol, digoxigenin, fluorescein, and rhodamine, all of which are available as NHS-esters that may be reacted with alkynylamino-derivatized terminators. These reagents as well as antibody capture agents for these compounds are available for Molecular Probes, Inc. (Eugene, Oreg.).

### Exemolary Hybridization-Based Assays

There are many hybridization-based assays that comprise a hybridization step that forms a structure or complex with a target polynucleotide, such as a fragment of genomic DNA, and an enzymatic processing step in which one or more enzymes either recognize such structure or complex as a substrate or are prevented from recognizing a substrate because it is protected by such structure or complex. In particular, such assays are widely used in multiplexed formats to simultaneously analyze DNA samples at multiple loci, e.g. allele-specific muliplex PCR, arrayed primer extension (APEX) technology, solution phase primer extension or ligation assays, and the like, described in the following exemplary references: Syvanen, Nature Genetics Supplement, 37: S5-S10 (2005); Shumaker et al, Hum. Mut., 7: 346-354 (1996); Huang et al, U.S. Pat. Nos. 6,709,816 and 6,287,778; Fan et al, U.S. patent publication 2003/0003490; Gunderson et al, U.S. patent publication 2005/0037393; Hardenbol et al, Nature Biotechnology, 21: 673-678 (2003); Nilsson et al, Science, 265: 2085-2088 (1994); Baner et al, Nucleic Acids Research, 26: 5073-5078 (1998); Lizardi et al, Nat. Genet., 19: 225-232 (1998); Gerry et al, J. Mol. Biol., 292: 251-262 (1999); Fan et al, Genome Research, 10: 853-860 (2000); International patent publications WO 2002/57491 and WO 2000/58516; U.S. Pat. Nos. 6,506,594 and 4,883,750; and the like.

In one aspect, hybridization-based assays include circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP. 4-262799; rolling circle probes being disclosed in Aono et al, JP-4-262799; Lizardi, U.S. Pat. Nos. 5,854,033; 6,183,960; 6,344,239; molecular inversion probes being disclosed in Hardenbol et al (cited above) and in Willis et al, U.S. Pat. No. 6,858,412; and linear amplification molecules being disclosed in Faham et al, U.S. patent publication 2003/0104459; all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatatemers, and the like, with an exonuclease, such as exonuclease I.

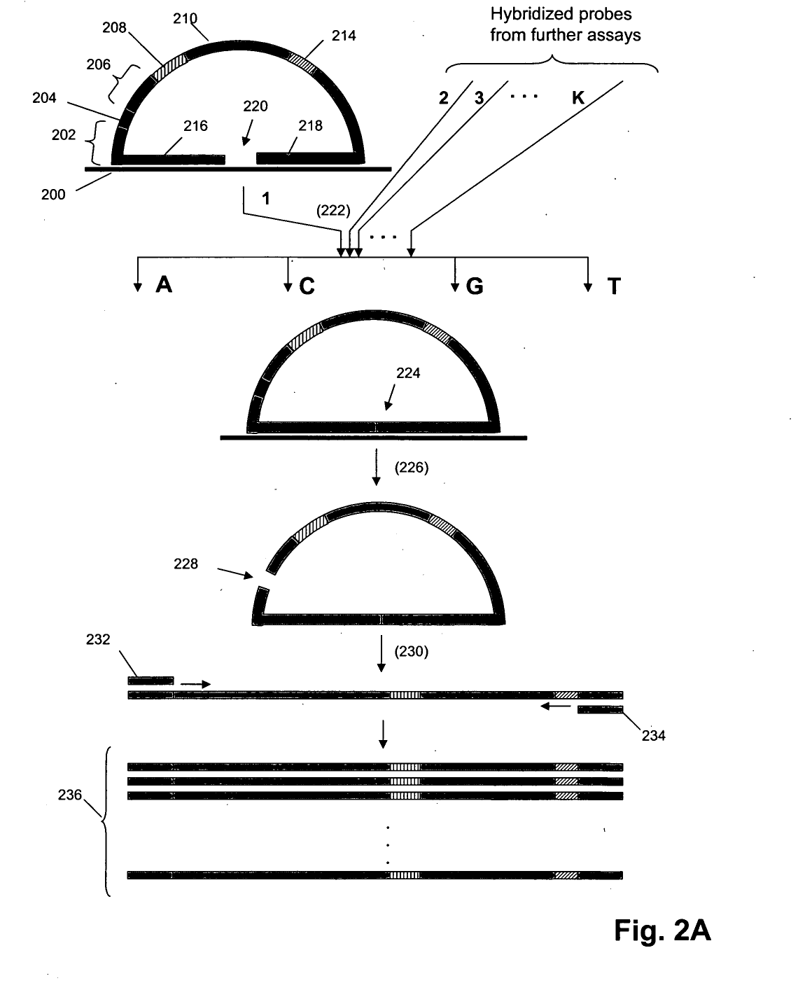

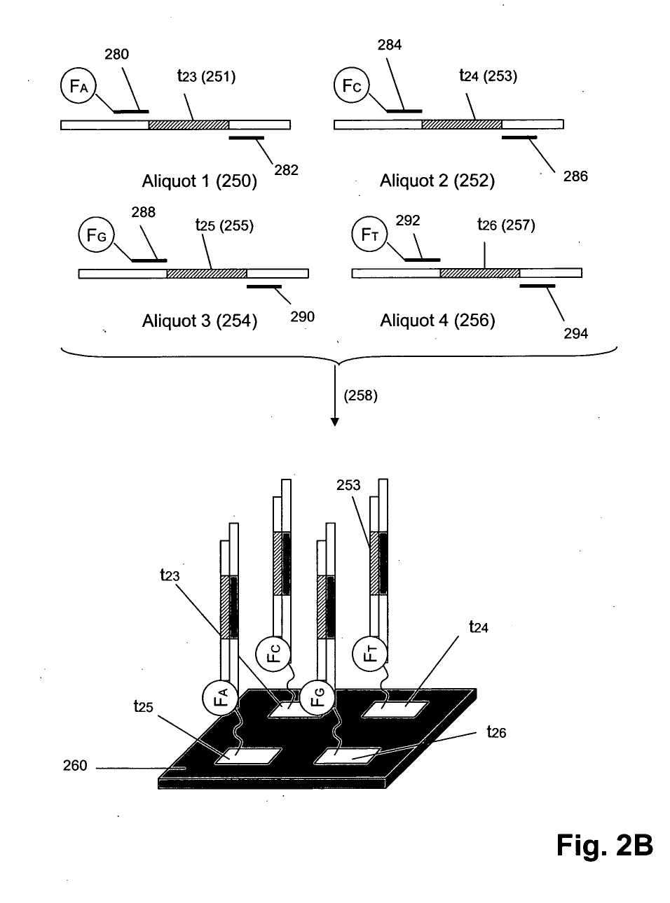

FIGS. 2A-2B illustrate a molecular inversion probe and how such probes are used in accordance with the invention. As illustrated in FIG. 2A, a panel (or set) of linear probe molecules (illustrated for sample 1) is combined separately with each of genomic samples 1 through K under conditions that permit target-specific region 1 (216) and target-specific region 2 (218) to form stable duplexes with complementary regions of respective target polynucleotides (200) in each of the separate assays. The ends of the target-specific regions may abut one another (being separated by a “nick”) or there may be a gap (220) of several (e.g. 1-10 nucleotides) between them, depending on the embodiment of the molecular inversion probe assay employed. After sufficient time has been allowed for specific hybridization, assay mixtures, 1 through K, are combined (222) for subsequent enzymatic processing steps. In one version of molecular inversion probe assays, the combined mixtures are separated into four aliquots for separate enzymatic treatment (A, C, G, or T extensions, respectively, followed by ligation and exonuclease treatment), after which the aliquots are recombined for specifically hybridizing labeled oligonucletide tags to a readout platform.

After hybridization of the target-specific regions, the ends of the two target specific regions are covalently linked by way of a ligation reaction or an extension reaction followed by a ligation reaction, i.e. a so-called “gap-filling” reaction. The latter reaction is carried out by extending with a DNA polymerase a free 3′ end of one of the target-specific regions so that the extended end abuts the end of the other target-specific region, which has a 5′ phosphate, or like group, to permit ligation. In one aspect, molecular inversion probes each have a structure as illustrated in FIG. 2A. Besides target-specific regions (216 and 218), in sequence such a probe may include first primer binding site (202), cleavage site (204), second primer binding site (206), first tag-adjacent sequences (208) (usually restriction endonuclease sites and/or primer binding sites) for tailoring one end of a labeled target sequence containing oligonucleotide tag (210), and second tag-adjacent sequences (214) for tailoring the other end of a labeled target sequence. Alternatively, cleavage-site (204) may be added at a later step by amplification using a primer containing such a cleavage site. In operation, after specific hybridization of the target-specific regions and their ligation, the reaction mixture is treated with a single stranded exonuclease that preferentially digests all single stranded nucleic acids, except circularized probes. After such treatment, circularized probes are treated (226) with a cleaving agent that cleaves the probe between primer (202) and primer (206) so that the structure is linearized (230). Cleavage site (204) and its corresponding cleaving agent is a design choice for one of ordinary skill in the art. In one aspect, cleavage site (204) is a segment containing a sequence of uracil-containing nucleotides and the cleavage agent is treatment with uracil-DNA glycosylase followed by heating. After the circularized probes are opened, the linear product is amplified, e.g. by PCR using primers (232) and (234), to form amplicons (236). Oligonucleotide tags (210) are then amplified and labeled for specific hybridization to a microarray of tag complements, e.g. a GenFlex array (Affymetrix, Santa Clara, Calif.); or the like.

FIG. 2B illustrates a labeling scheme for generating a different signal, e.g. a different fluorescent signal, for each of four alternative nucleotides at a locus, where a microarray is used to detect signals generated by labeled oligonucleotide tags. In this scheme, amplicons (236) from each of the four aliquots (250) through (256) (illustrated with oligonucleotide tags t₂₃ (251), t₂₄ (253), t₂₅ (255), and t₂₆ (257), respectively) are combined with primer pairs (280) and (282), (284) and (286), (288) and (290), and (292) and (294), respectively, and amplified, e.g. by PCR. Primers (280), (284), (288), and (292) have attached spectrally resolvable fluorescent dyes, F_{A}, F_{C}, F_{G}, and F_{T}, respectively. After amplification, the respective products are combined, denatured, and applied (258) to microarray (260) so that each oligonucleotide tag specifically hybridizes with its tag complement. Fluorescent signals from the features of microarray (260) are then collected and analyzed using conventional instrumentation, e.g. GeneChip® Scanner 3000 (Affymetrix, Santa Clara, Calif.), or like instrument.

### Hybridization-Based Assays Employing Solid Phase Supports

Methods of conducting multiplexed hybridization-based assays using microarrays, and like platforms, suitable for the present invention are well known in the art. Guidance for selecting conditions and materials for applying labeled sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about IM, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to about 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled target sequence to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂, PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3M TMACL (Tetramethylammonium. Chloride), 50 mM MES ((2-[N-Morpholino]ethanesulfonic acid) Sodium Salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip™ Software (Affymetrix) or like software may be used to convert the image files into digitized files for further data analysis.

### Sample Preparation

Samples or specimens containing target polynucleotides, such as fragments of genomic DNA, may come from a wide variety of sources for use with the present invention, including cell cultures, animal or plant tissues, patient biopsies, environmental samples, or the like. Samples are prepared for assays of the invention using conventional techniques, which typically depend on the source from which a sample or specimen is taken.

Prior to carrying out reactions on a sample, it will often be desirable to perform one or more ample preparation operations upon the sample. Typically, these sample preparation operations will nclude such manipulations as extraction of intracellular material, e.g., nucleic acids from whole cell amples, viruses and the like.

For those embodiments where whole cells, viruses or other tissue samples are being analyzed, t will typically be necessary to extract the nucleic acids from the cells or viruses, prior to continuing ith the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

In some applications, such as measuring target polynucleotides in rare cells from a patient's blood, an enrichment step may be carried out prior to conducting an assay, such as by immunomagnetic isolation. Such isolation or enrichment may be carried out using a variety of techniques and materials known in the art, as disclosed in the following representative references that are incorporated by reference: Terstappen et al, U.S. Pat. No. 6,365,362; Terstappen et al, U.S. Pat. No. 5,646,001; Rohr et al, U.S. Pat. No. 5,998,224; Kausch et al, U.S. Pat. No. 5,665,582; Kresse et al, U.S. Pat. No. 6,048,515; Kausch et al, U.S. Pat. No. 5,508,164; Miltenyi et al, U.S. Pat. No. 5,691,208; Molday, U.S. Pat. No. 4,452,773; Kronick, U.S. Pat. No. 4,375,407; Radbruch et al, chapter 23, in Methods in Cell Biology, Vol, 42 (Academic Press, New York, 1994); Uhlen et al, Advances in Biomagnetic Separation (Eaton Publishing, Natick, 1994); Safarik et al, J. Chromatography B, 722: 33-53 (1999); Miltenyi et al, Cytometry, 11: 231-238 (1990); Nakamura et al, Biotechnol. Prog., 17: 1145-1155 (2001); Moreno et al, Urology, 58: 386-392 (2001); Racila et al, Proc. Natl. Acad. Sci., 95: 4589-4594 (1998); Zigeuner et al, J. Urology, 169: 701-705 (2003); Ghossein et al, Seminars in Surgical Oncology, 20: 304-311 (2001).

In one aspect, genomic DNA for analysis is obtained using standard commercially available DNA extraction kits, e.g. PureGene® DNA Isolation Kit (Gentra Systems, Minneapolis, Minn.). In another aspect, for assaying human genomic DNA with a multiplex hybridization-based assay containing from about 1000 to 50,000 probes, a DNA sample may be used having an amount within the range of from about 200 ng to about 1 μg. When sample material is scarce, prior to assaying, sample DNA may be amplified by whole genome amplification, or like technique, to increase the total amount of DNA available for performing an assay on. Several whole genome, or partial genome, amplification techniques are known in the art, such as the following which are incorporated by reference: Telenius et al, Genormics, 13: 718-725 (1992); Cheung et al, Proc. Natl. Acad. Sci., 93: 14676-14679 (1996); Dean et al, Genome Research, 11: 1095-1099 (2001); U.S. Pat. Nos. 6,124,120; 6,280,949; 6,617,137; and the like.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

What is claimed is the following:

1. A method of simultaneously analyzing a plurality of genomes to obtain sequence information at one or more loci in each genome, the method comprising the steps of:
providing for each genome a set of probes, each probe within a set being specific for a locus of the genome;
separately hybridizing each set of probes with its respective genome to form probe-genome complexes in separate reaction mixtures;
combining the separate reaction mixtures and enzymatically treating the probe-genome complexes to form amplifiable probes;
amplifying and labeling the amplifiable probes to form labeled probes, so that for each different locus of each different genome there is a unique labeled probe; and
specifically hybridizing the labeled probes to their respective complements on a microarray so that the presence or absence of a labeled probe specifically hybridized to the microarray is indicative of sequence information of each of the one or more loci of each genome in the plurality.

2. The method of claim 1 wherein each of said probes from each of said sets contains an oligonucleotide tag.

3. The method of claim 2 wherein said step of amplifying and labeling includes amplifying and labeling said oligonucleotide tags in said probes so that for each different locus of each different genome there is a unique labeled oligonucleotide tag.

4. The method of claim 3 wherein said step of specifically hybridizing includes specifically hybridizing said labeled oligonucleotide tags to their respective tag complements on said microarray so that the presence or absence of a labeled oligonucleotide tag on said microarray is indicative of said sequence information on each of said one or more loci of each of said genomes.

5. The method of claim 4 wherein each labeled oligonucleotide tag has a nucleotide sequence and a label for generating an optical signal and wherein each of said loci of each of said genomes is identified by the nucleotide sequence and label of at least one labeled oligonucleotide tag.

6. The method of claim 5 wherein said oligonucleotide tags for different loci are different and wherein at at least one locus of each of said plurality of said genomes more than one oligonucleotide tags for the same locus of different said genomes are the same.

7. The method of claim 6 wherein said more than one of said oligonucleotide tags that are the same are each labeled with a different spectrally resolvable fluorescent dye.

8. The method of claim 5 wherein said sequence information includes identity of single nucleotide polymorphisms, nucleotide insertions, or nucleotide deletions at said loci.

9. The method of claim 8 wherein said step of enzymatically treating said probe-genome complexes includes extending a probe with a DNA polymerase in a template-driven reaction.

10. The method of claim 8 wherein said step of enzymatically treating said probe-genome complex includes ligating a first component of a probe to a second component of the probe in a template-driven reaction.

11. The method of claim 8 wherein each of said probes is a circularizable probe.

12. The method of claim 11 wherein each of said circularizable probes is a molecular inversion probe and wherein each of said amplifiable probes is a circularized molecular inversion probe.

13. A method of simultaneously analyzing a plurality of samples to determine the presence or quantity of one or more analytes, the method comprising the steps of:
providing for each sample a set of probes including at least one probe for each analyte, each probe within a set being specific for at least one of the analytes and each probe within a set having an oligonucleotide tag attached;
separately reacting each set of probes with its respective sample to form probe-analyte complexes in separate reaction mixtures;
combining the separate reaction mixtures and enzymatically treating the probe-analyte complexes to form amplifiable probes;
amplifying and labeling the amplifiable probes to form labeled oligonucleotide tags, so that for each different analyte of each different sample there is a unique labeled oligonucleotide tag; and
specifically hybridizing the labeled oligonucleotide tags to their respective complements on a microarray so that the presence or absence of a labeled oligonucleotide tag specifically hybridized to the microarray is indicative of the presence or quantity of each of the one or more analytes of each sample in the plurality.
